(19) United States
(12) Patent Application Publication
(10) Pub. No.: US 2023/0181519 A1
(43) Pub. Date: Jun. 15, 2023

(54) Compositions And Methods For Treating Nicotine-Related Disorders And Symptoms Using Dihydromyricetin

(71) Applicant: Jie Wu, Avondale, AZ (US)

(72) Inventor: Jie Wu, Avondale, AZ (US)

(21) Appl. No.: 17/548,550

(22) Filed: Dec. 12, 2021

(51) Int. Cl. A61K 31/353; A61P 25/34

(52) U.S. Cl. CPC A61K 31/353; A61P 25/34

(57) ABSTRACT

The invention relates to pharmaceutical compositions comprising dihydromyricetin, methods for making such compositions, and methods for using such compositions to treat nicotine-related disorders and habits, such as nicotine addiction, nicotine withdrawal symptoms and the reduction and/or cessation of smoking, vaping, patch, and/or chewing tobacco products.

### FIELD OF THE INVENTION

The invention relates to compositions comprising dihydromyricetin, methods for making such compositions, and methods for using such compositions.

### BACKGROUND OF THE INVENTION

Nicotine-related disorders and symptoms include several neurological disorders and symptoms such as nicotine habits and addiction from smoking, vaping, patch, and/or chewing tobacco products. Methods for treating such disorders and symptoms have not met with universal success and new methods and new pharmaceutical compositions are needed to treat nicotine-related disorders and symptoms.

### SUMMARY OF THE INVENTION

The subject matter of this invention in certain embodiments concerns dihydromyricetin (“DHM”) and its effects on nicotinic acetylcholine receptors (“nAChRs”). Preferred embodiments of this invention include pharmaceutical and other compositions that comprise DHM, a bioflavonoid that can be isolated from Hovenia dulcis. Preferred embodiments of the pharmaceutical compositions of this invention can be used in methods of the invention to inhibit nicotine receptor function and nicotine reward/dependence behaviors in persons and animals, such as mammals. This invention also includes pharmaceutical compositions and methods for using them such as in methods of treating disorders that can aid in the reduction and/or stopping of nicotine use, such as the reduction and/or stopping of the use of nicotine in smoking, vaping, patch, and/or chewing tobacco and other uses of nicotine that may be habit forming or addicting.

In the most preferred embodiments of this invention, a composition comprising DHM is used for treating tobacco addiction or nicotine addiction, palliating nicotine withdrawal symptoms or facilitating smoking cessation. The method includes administering the composition comprising DHM and then, optionally, determining whether the tobacco addiction, nicotine withdrawal symptoms or smoking has been affected. In especially referred embodiments of methods, DHM is formulated in an orally available form and information is provided to a user that concerns DHM and the treatment of DHM for, or effect on, tobacco addiction, nicotine addiction, palliating nicotine withdrawal symptoms, inhibiting nicotine reward, inhibiting nicotine dependence behaviors, inhibiting nicotine receptor function, reducing nicotine patch use, or facilitating cessation of smoking.

In addition, in certain preferred embodiments the pharmaceutical compositions of this invention can be used in methods for improving the quality and/or quantity of sleep of a patient.

### BRIEF DESCRIPTION OF THE DRAWINGS

FIG. 1 shows effects of dihydromyricetin (“DHM”) on transfected α4β2-nAChRs in human SH-EP1 cells.

FIG. 2 shows effects of DHM on mouse ventral tegmental area (“VTA”) dopamine (“DA”) neuronal firing in VTA slices.

FIG. 3 shows effects of the systematic injection of nicotine (“NIC”) (0.5 mg/kg, i.v.) on VTA DA neuronal firing rate in anesthetized mice.

FIG. 4 shows effects of DHM on VTA DA neuronal firing in anesthetized mice.

FIG. 5 shows effects of DHM on NIC-induced conditioned-place preference (“CPP”).

FIG. 6 shows NIC-induced behavioral sensitization in mice.

FIG. 7 shows effects of DHM on NIC-induced behavioral sensitization and a comparison of NIC sensitization between the NIC group and NIC+DHM group.

### DETAILED DESCRIPTION OF THE INVENTION

The invention provides certain embodiments that include compositions that include dihydromyricetin (“DHM”), methods of using such compositions, methods of making such compositions, and methods of treating problems and disorders related to nicotine and DHM.

The present inventors have determined that DHM is an effective inhibitor of nicotine receptor function and nicotine reward/dependence behaviors. Moreover, when delivered systemically, DHM can block the effects of nicotine on the central nervous system (CNS), indicating that this drug is able to cross the blood-brain barrier and access sites in the brain. Such nicotinic inhibitors with CNS activity have been shown to be useful treatments for neuropsychiatric disorders and for promoting smoking cessation. Preferably, the compounds (DHM, or a pharmaceutically acceptable salt thereof, or an analog, prodrug or metabolite) and composition embodiments (e.g., pharmaceutical compositions) of this invention are administered to treat a patient suffering from a problem or disorder associated with nicotine use, such as nicotine smoking addiction.

In another embodiment of the method of the present invention, DHM or a pharmaceutically acceptable salt thereof, is administered to a patient for treatment or prevention of nicotine addiction. DHM or a pharmaceutically acceptable salt thereof can be administered with another pharmacologically active compound to treat or prevent nicotine addiction. For example, DHM or a pharmaceutically acceptable salt thereof can be administered to a patient in combination with nicotine (e.g., nicotine can be transdermally administered by application of a nicotine patch).

Mammalian species which benefit from the disclosed methods of treatment include, and are not limited to, apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. The term “patient” is intended to include such human and non-human mammalian species.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19th ed., 1995) describes formulations that can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile pharmacologically acceptable salts can be prepared in water suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, suitably mixed with a surfactant, such as hydroxypropylcellulose. Dispersions can also be prepared in glycerol, liquid polyethylene glycols, and mixtures thereof and in oils. Under ordinary conditions of storage and use, these preparations for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation and/or treatment in question. DHM, or a pharmaceutically acceptable salt thereof (or pharmaceutical compositions containing DHM or a pharmaceutically acceptable salt thereof), can be administered to a patient by any route that results in prevention or alleviation of symptoms associated with the particular neurological condition. For example, as described in more detail below, DHM or a pharmaceutically acceptable salt thereof can be administered parenterally, intravenously (I.V.), intramuscularly (I.M.), subcutaneously (S.C.), intradermally (I.D.), orally, intranasally, etc. Examples of intranasal administration can be by means of a spray, drops, powder or gel. However, other means of drug administrations are well within the scope of the present invention.

The pharmaceutical compositions disclosed herein may be orally administered, for example, with an inert diluent or with an assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of the unit. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, DHM or a pharmaceutically acceptable salt thereof may be incorporated into sustained-release preparation and formulations.

DHM may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases of injection, the form must be sterile and must be fluid to the extent that easy syringe-ability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating DHM, or a pharmaceutically acceptable salt thereof, in the required amount in the appropriate solvent with other various ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Examples of “pharmaceutically acceptable carriers” include any and all solvents, dispersion media, coatings, antibacterial and antifungal agents, isotonic and absorption delaying agents and the like. The use of such media and agents for pharmaceutical active substances is well known in the art. In one embodiment, the pharmaceutically acceptable carrier is a sterile, fluid (e.g., liquid or gas) preparation rendering the pharmaceutical composition suitable for injection or inhalation. Except insofar as any conventional media or agent is incompatible with the active ingredient, its use in the therapeutic compositions is contemplated. Supplementary active ingredients can also be incorporated into the compositions.

For oral prophylaxis, DHM or a pharmaceutically acceptable salt thereof, may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, DHM or a pharmaceutically acceptable salt thereof, may be incorporated into an antiseptic wash containing sodium borate, glycerin and potassium bicarbonate. DHM or a pharmaceutically acceptable salt thereof may also be dispersed in dentifrices, including: gels, pastes, powders and slurries. DHM or a pharmaceutically acceptable salt thereof may be added in a therapeutically effective amount to a paste dentifrice that may include water, binders, abrasives, flavoring agents, foaming agents, and humectants.

The phrase “pharmaceutically acceptable” refers to molecular entities and compositions that do not produce an allergic or similar untoward reaction when administered to a human. The preparation of an aqueous composition that contains an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

The composition of the present invention can be formulated in a neutral or salt form. Pharmaceutically-acceptable salts, include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, Remington's Pharmaceutical Sciences). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards (or some other relevant agency or standard).

According to the therapeutic methods of the present invention, DHM or a pharmaceutically acceptable salt thereof, is administered (e.g., brought into contact with nAChRs) and dosed in accordance with good medical practice, taking into account the clinical condition of the individual patient, the site and method of administration, scheduling of administration, patient age, sex, body weight, and other factors known to medical practitioners. The pharmaceutically or therapeutically “effective amount” for purposes herein is thus determined by such considerations as are known in the art. For therapeutic methods, the amount of DHM, or a pharmaceutically acceptable salt thereof, must be effective to achieve improvement including but not limited to total prevention and to improved survival rate or more rapid recovery, or improvement or elimination of symptoms associated with the particular neurological condition, such as nicotine habits or addiction, and other indicators as are selected as appropriate measures by those skilled in the art. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration. Optionally, the therapeutic methods of the invention further comprise diagnosis of the patient with a neurological condition by a medical practitioner (e.g., a medical doctor, veterinarian, or other clinician). The therapeutic methods may further comprise evaluating the patient for one or more symptoms associated with the neurological condition before and/or after administration of DHM or a pharmaceutically acceptable salt thereof.

As used herein, in preferred embodiments an “effective amount” of DHM is an amount that results in the desired effect as compared to a control—an amount that treats, inhibits, reduces and/or reverses a nicotine-related affect. In preferred embodiments, a “therapeutically effective amount” of DHM is a quantity sufficient to, when administered to a subject, treat, inhibit, reduce and/or reverse a nicotine-related effect in the subject such that the condition of the subject is an observable improvement as compared to the condition of the subject prior to the treatment or as compared to a control subject. Also, as used herein, a “therapeutically effective amount” of DHM is an amount which when administered to the subject treats a given clinical condition, e.g., nicotine addiction, in the subject as compared to a control. Typically, therapeutically effective amounts of DHM can be orally or intravenously administered daily at a dosage of about 0.002 to about 200 mg/kg, preferably about 0.1 to about 100 mg/kg, e.g., about 1 mg/kg of body weight.

In these preferred embodiments, a dose of 0.01 to 10 mg/kg in divided doses one to four times a day, or in sustained release formulation will be effective in obtaining the desired pharmacological effect. It will be understood, however, that the specific dose levels for any particular subject will depend upon a variety of factors including the activity of the specific compound employed, the age, body weight, general health, sex, diet, time of administration, route of administration, and rate of excretion, drug combination and the severity of the particular disease and/or condition. Frequency of dosage may also vary depending on the particular disease and/or condition treated. It will also be appreciated that the effective dosage for treatment may increase or decrease over the course of a particular treatment. Changes in dosage may result and become apparent by standard diagnostic assays in clinical techniques known in the art.

In some instances, chronic administration may be required. Effective amounts and therapeutically effective amounts of DHM may be readily determined by one of ordinary skill by routine methods known in the art. In some embodiments, an effective amount of DHM may be administered in the form of a foodstuff, such as a beverage. In some embodiments, an effective amount of DHM may be administered in the form of a chewing gum composition.

In these preferred embodiments, the pharmaceutical formulations of the invention comprise a divided dose or a single dose of DHM and may be prepared in a unit-dosage form and/or packaging appropriate for the desired mode of administration. The pharmaceutical formulations of the present invention may be administered for therapy by any suitable route including oral, rectal, nasal, topical (including buccal and sublingual), dermal, mucosal, vaginal and parenteral (including subcutaneous, intramuscular, intravenous and intradermal). It will be appreciated that the preferred route will vary with the condition and age of the recipient, the nature of the condition to be treated. For example, in some embodiments, a therapeutically effective amount of DHM may be administered to a subject in the form of a transdermal patch or an effervescent tablet (e.g., a tablet comprising an effective amount of DHM, a carbonate salt, such as sodium bicarbonate, and an acidic material, such as citric acid which results in effervescence when dissolved in a liquid such as water).

In some embodiments, the unit dose of DHM for a human subject is about 50-70 mg. Thus, in some embodiments, foodstuffs, transdermal patches, chewing gums, and/or effervescent tablets according to the present invention comprise about 50-70 mg per unit.

The methods and compositions of the invention may incorporate additional pharmacologically active agents (such as for adjunctive therapy), in addition to DHM or a pharmaceutically acceptable salt thereof. For example, the additional pharmacologically active agent can be co-administered consecutively or simultaneously (e.g., in the same formulation or different formulations).

In one embodiment, the additional pharmacologically active agent is an nAChR modulator, such as an inhibitor of nAChR activity. Preferably, the agent is a selective inhibitor.

As used herein, the term “treatment” or grammatical variations thereof is intended to mean reducing (e.g., lessening or eliminating) or preventing of one or more symptoms associated with a particular neurological disorder characterized by dysfunction (e.g., overactivation or overexpression) of nAChR.

As used herein, “activity” of a nAChR refers to any activity characteristic of a nAChR. Such activity can typically be measured by one or more in vitro methods, and frequently corresponds to an in vivo activity of a nAChR. Thus, the terms “function” and “activity” with respect to nicotine AChR means that the receptor channel is able to provide for and regulate entry of nicotinic AChR-permeable ions, such as, for example, Na+, K+, Ca2+, or Ba2+, in response to a stimulus and/or bind ligands with affinity for the receptor. Preferably, such nicotinic AChR activity is distinguishable, such as by electrophysiological, pharmacological, and other means known to those of skill in the art, from the endogenous nicotinic AChR activity that may be produced by the host cell in the absence of DHM or a pharmaceutically acceptable salt thereof. As used herein, the term “inhibit” with respect to nAChR activity is intended to include partial or complete inhibition of nAChR activity.

In another aspect, the present invention pertains to methods for selectively inhibiting nicotinic acetylcholine receptors by contacting an effective amount of DHM, or a pharmaceutically acceptable salt thereof, to the receptor. The method can be carried out in vivo or in vitro. For example, an effective amount of DHM, or a pharmaceutically acceptable salt thereof, can be administered in vivo to a patient in need thereof. Alternatively, an effective amount of DHM, or a pharmaceutically acceptable salt thereof, can be contacted in vitro to nicotine acetylcholine receptors or to cells that naturally or recombinantly express nicotine acetylcholine receptors. Optionally, the method further comprises determining nAChR activity before, during, or after the DHM or a pharmaceutically acceptable salt thereof is contacted with the nAChR. The effect of DHM or a pharmaceutically acceptable salt thereof on nAChR can be determined by comparison of the change in receptor function (e.g., by electrophysiological recordings). The nAChR can be mammalian, such as human. Examples of host cells appropriate for recombinant expression of nAChR include, but are not limited to, bacterial cells (e.g., Escherichia coli), yeast cells (e.g., methylotrophic yeast cells, such as Pichia pastoris), and mammalian cells (e.g., SH-EP1, HEK 293, CHO and Ltk− cells). The cells to which DHM or a pharmaceutically acceptable salt thereof may be contacted in vitro include isolated cells, cell cultures, cell lines, tissues (e.g., tissue cultures), etc.

In certain preferred embodiments of this invention, particular methods for using (e.g., treating, reducing, inhibiting) DHM and pharmaceutical compositions comprising DHM, include (1) a method of treating nicotine addiction in a person or animal, comprising administering a therapeutically effective amount of a composition comprising dihydromyricetin, or a pharmaceutically acceptable salt thereof, to the person or animal, and then determining whether the nicotine addiction has been reduced and/or eliminated; (2) a method of reducing the desire to smoke, vape, or chew tobacco in a person or animal, comprising administering a therapeutically effective amount of a composition comprising dihydromyricetin, or a pharmaceutically acceptable salt thereof, to the person or animal, and then determining whether the desire to smoke, vape, or chew tobacco has been inhibited; (3) a method of inhibiting nicotine receptor function in a person or animal, comprising administering a therapeutically effective amount of a composition comprising dihydromyricetin, or a pharmaceutically acceptable salt thereof, to the person or animal, and then determining whether the nicotine receptor function has been inhibited; and (4) a method of inhibiting nicotine reward and/or dependence behaviors in a person or animal, comprising administering a therapeutically effective amount of a composition comprising dihydromyricetin, or a pharmaceutically acceptable salt thereof, to the person or animal, and then determining whether the nicotine reward and/or dependence behavior has been inhibited. For these methods, in certain embodiments the pharmaceutical composition can also comprise a pharmaceutically acceptable carrier. In other embodiments, the pharmaceutical composition is incorporated into a sustained, controlled or delayed release preparation.

Particularly preferred embodiments of this invention include a method for treating, inhibiting and/or reducing the effects of nicotine and/or a symptom of nicotine withdrawal in a person or animal, comprising administering dihydromyricetin to the patient or animal. Other particularly preferred embodiments of this invention include a method for treating tobacco addiction or nicotine addiction, palliating nicotine withdrawal symptoms or facilitating smoking cessation comprising administering a therapeutically effective amount of dihydromyricetin.

Throughout the subject application, DHM may be substituted with a metabolite or chemical analog in connection with the compound, composition, and methods of the present invention. As used herein, the term “analogs” refers to compounds which are substantially the same as another compound but which may have been modified by, for example, adding side groups, oxidation or reduction of the parent structure. Analogs of the exemplified compounds can be readily prepared using commonly known standard reactions. These standard reactions include, but are not limited to, hydrogenation, alkylation, acetylation, and acidification reactions.

As used herein, the term “additional pharmacologically active agent” refers to any agent, such as a drug, capable of having a physiologic effect (e.g., a therapeutic or prophylactic effect) on prokaryotic or eukaryotic cells, in vivo or in vitro, including, but without limitation, chemotherapeutics, toxins, radiotherapeutics, radiosensitizing agents, gene therapy vectors, antisense nucleic acid constructs or small interfering RNA, imaging agents, diagnostic agents, agents known to interact with an intracellular protein, polypeptides, and polynucleotides.

The additional pharmacologically active agent can be selected from a variety of known classes of drugs, including, for example, analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyraid calcitonin and biphosphonates, prostaglandins, radiopharmaceuticals, hormones, sex hormones (including steroids), time release binders, anti-allergic agents, stimulants and anoretics, steroids, sympathomimetics, thyroid agents, vaccines, vasodilators, and xanthines.

The additional pharmacologically active agent need not be a therapeutic agent. For example, the agent may be cytotoxic to the local cells to which it is delivered but have an overall beneficial effect on the subject. Further, the agent may be a diagnostic agent with no direct therapeutic activity per se, such as a contrast agent for bioimaging.

In certain embodiments of methods of this invention, DHM can be administered as an isolated compound in a pharmaceutical composition, or administered in a pharmaceutically acceptable carrier and with or without other pharmaceutical excipients as a pharmaceutical composition of this invention. Optionally, DHM can be administered with other pharmacologically active agents. These can include many different compounds, such as nicotinic acethylcholine receptor agonists, antagonists, or mixed agonists/antagonists. It can also include compounds that are used to treat nicotinic disorders, such as compounds that are used to reduce and/or stop smoking, vaping or chewing tobacco and other nicotine containing products.

In other aspects, certain embodiments of the present invention concerns a compound comprising DHM or a pharmaceutically acceptable salt thereof; and pharmaceutical compositions containing DHM, or a pharmaceutically acceptable salt thereof, and a pharmaceutically acceptable carrier.

In another aspect, certain embodiments of the invention pertain to methods for selectively inhibiting nicotinic acetylcholine receptors and/or their function. In still another aspect, certain embodiments of the invention pertain to methods for inhibiting nicotine reward/dependence behaviors. Certain embodiments of these methods of inhibiting such receptors, functions and/or behaviors are by contacting an effective amount (e.g., a therapeutically effective amount) of DHM, or a pharmaceutically acceptable salt thereof, in a pharmaceutical composition, with or without other pharmaceutical excipients, to the receptor. The methods can be carried out in vivo or in vitro. For example, an effective amount of DHM, or a pharmaceutically acceptable salt thereof, can be administered in vivo to a patient in need thereof, in order to treat a problem or neurological condition, such as a nicotine habit or a nicotine addiction. Alternatively, an effective amount (e.g., a therapeutically effective amount) of DHM, or a pharmaceutically acceptable salt thereof, can be contacted in vitro to isolated nicotine acetylcholine receptors or such receptors in cells, tissues, organs or in animal experiments.

In certain preferred embodiments the pharmaceutical compositions of this invention can be used in methods for improving the quality and/or quantity of sleep of a patient. Improved quality of sleep can be characterized in several different ways, including, but not limited to (1) falling asleep soon after going to bed (e.g., less than 30 minutes); (2) typically sleeping straight through the night and waking up no more than once per night; (3) sleeping the recommended hours for the applicable age group; (4) falling back asleep within 20 minutes or less if wake up; and/or (5) feeling rested, restored, and energized upon waking up in the morning. Improved quantity of sleep can be characterized in several different ways also, including, but not limited to measuring the amount of time of sleep.

These methods of improving sleep can include administering the pharmaceutical compositions of this invention before bedtime and/or with other dosing regimens. In addition, the improved sleep may be concomitant and/or simultaneous to using the pharmaceutical compositions in the other methods of this invention, such as treating nicotine addiction; reducing the desire to smoke, vape, nicotine patch or chew tobacco; inhibiting nicotine receptor function; inhibiting nicotine reward and/or dependence behaviors; treating tobacco addiction or nicotine addiction, palliating nicotine withdrawal symptoms or facilitating smoking cessation; and/or treating, inhibiting and/or reducing the effects of nicotine and/or a symptom of nicotine withdrawal.

The dose and dosing regimen of the pharmaceutical compositions of this invention to improve sleep can be determined for each patient by a person of skill in the art. Such a dose and dosing regimen may be the same, or substantially the same, as the dose and dosing regimen that is used in the other methods of this invention, such as treating nicotine addiction; reducing the desire to smoke, vape, nicotine patch or chew tobacco; inhibiting nicotine receptor function; inhibiting nicotine reward and/or dependence behaviors; treating tobacco addiction or nicotine addiction, palliating nicotine withdrawal symptoms or facilitating smoking cessation; and/or treating, inhibiting and/or reducing the effects of nicotine and/or a symptom of nicotine withdrawal.

The subject matter of this disclosure is now described with reference to the following examples. These examples are provided for the purpose of illustration only, and the subject matter is not limited to these examples, but rather encompasses all variations which are evident as a result of the teaching provided herein.

### Example 1

In this example, the effects of DHM on transfected α4β2-nAChRs in human SH-EP1 cells (a model system to study human acetylcholine (“ACh”) receptors) were measured. Traces of ACh-induced whole-cell currents co-applied with different concentrations of DHM were created and examined.

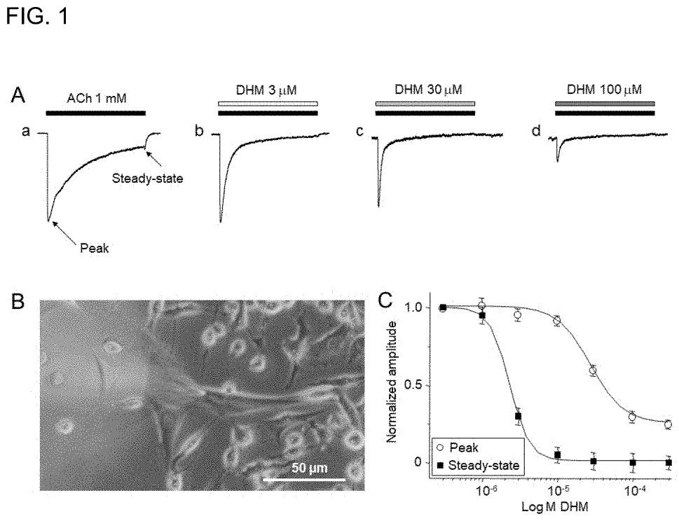

FIG. 1 shows effects of DHM on transfected α4β2-nAChRs in human SH-EP1 cells. Part A shows typical traces of nicotinic acetylcholine receptor—nAChR—ACh-induced whole-cell currents co-applied with different concentrations of DHM. Part B shows a phase-contract photo picture of cultured human SH-EP1 cells. Part C shows the concentration-response curves of the peak (black symbols) and steady-state (open symbols) of ACh responses with different concentrations of DHM.

### Example 2

In this example, the effects of DHM on mouse ventral tegmental area (“VTA”) dopamine (“DPA”) neuronal firing in slices were measured.

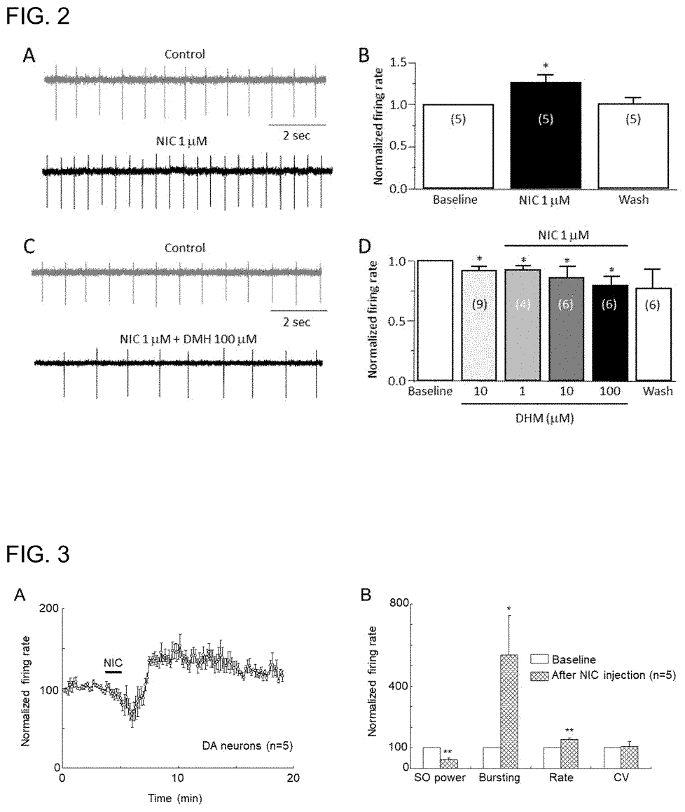

FIG. 2 shows the effects of DHM on mouse VTA DA neuronal firing in VTA slices. Part A shows typical spontaneous action potentials (“AP”) recorded from DA neurons of VTA slices using cell-attached recording mode (extracellular AP). An enhancement of AP firing rate was done by bath-applied nicotine (“NIC”). Part B shows a bar-graph that summarizes the enhanced effect of NIC on AP firing rate. Part C shows typical traces of the inhibitory effect of DHM on NIC's effect. Part D shows a summary of the effects of different concentrations of DHM on DA firing rate and on NIC's effects. Results showed that DHM itself reduced DA firing in the rat, and abolished NIC-induced enhancement of AP firing rate.

FIG. 3 shows effects of systematic injection of NIC (0.5 mg/kg, i.v.) on VTA DA neuronal firing rate in anesthetized mice. Part A is a time-frequency relationship graph that shows a dual-phase effect of NIC on DA neuronal firing, which is an initial reduction following by a long-lasting increase. Part B shows a statistic analysis of the systemic injection of NIC enhanced AP firing rate, the bursting firing, but a reduction in slow oscillations (SO).

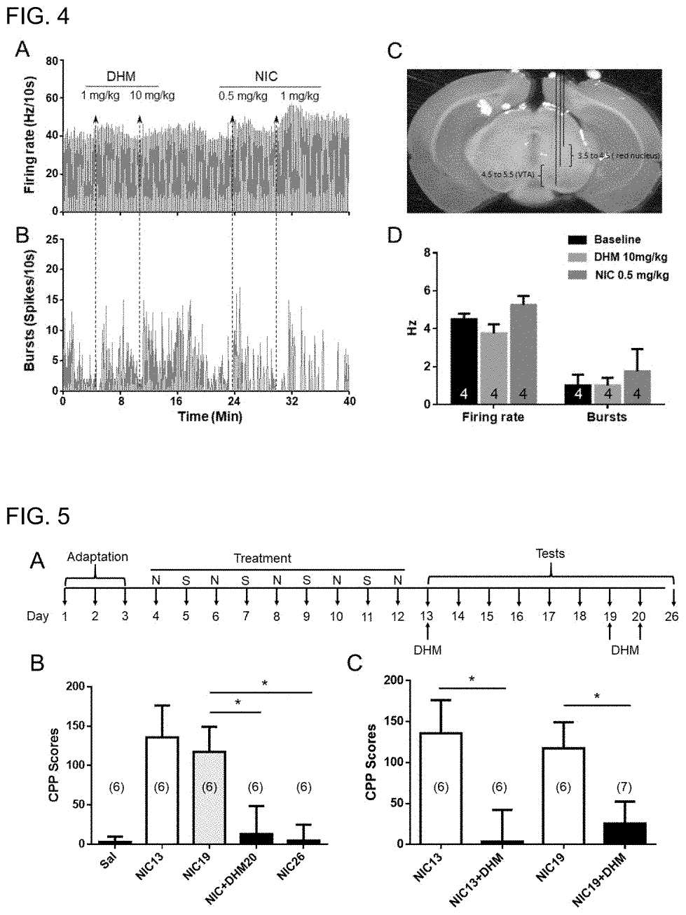

FIG. 4 shows effects of DHM on VTA DA neuronal firing in anesthetized mice. Part A is a time-frequency figure that shows the effects of DHM (1 and 10 mg/kg, i.v.) on spontaneous DA neuronal firing rate and bursting (Part B). Part C is after in vivo recording. The recording site was dyed using blue-sky dye, and confirmed by cutting brain sections that contained VTA. Part D is a bar-graph that summarizes the effects of DHM on NIC's effect, and showed that DHM reduced DA neuronal firing rate, and abolished the NIC-induced AP firing increase.

### Example 3

In this example, the effects of DHM on NIC-induced conditioned-place preference (“CCP”) were measured.

FIG. 5 shows effects of DHM on NIC-induced CPP. Part A is the CPP performance protocol. Part B shows that DHM abolished NIC-induced CPP after withdrawal 5 days of NIC. Part C shows DHM abolished NIC-induced CPP at test day (day 13) and after withdrawal 5 days (day 19). These results demonstrate that DHM significantly reduces NIC-induced CPP, and that DHM can be used to treat smoking habits and lead to smoking cessation.

### Example 4

In this example, the effects of DHM on NIC-induced behavioral sensitization in mice were measured.

FIG. 6 shows NIC-induced behavioral sensitization in mice. C57BL/J mice were repetitively treated with NIC (0.5 mg/kg, i.p.), and the local motor activities were measured. The results showed that with repetitive injections of NIC, mice motor activity was increased (total move distance), and after withdrawal at 5 and 10 days, the motor activity was still maintained at a high level, showing that the NIC-induced behavioral sensitization formed.

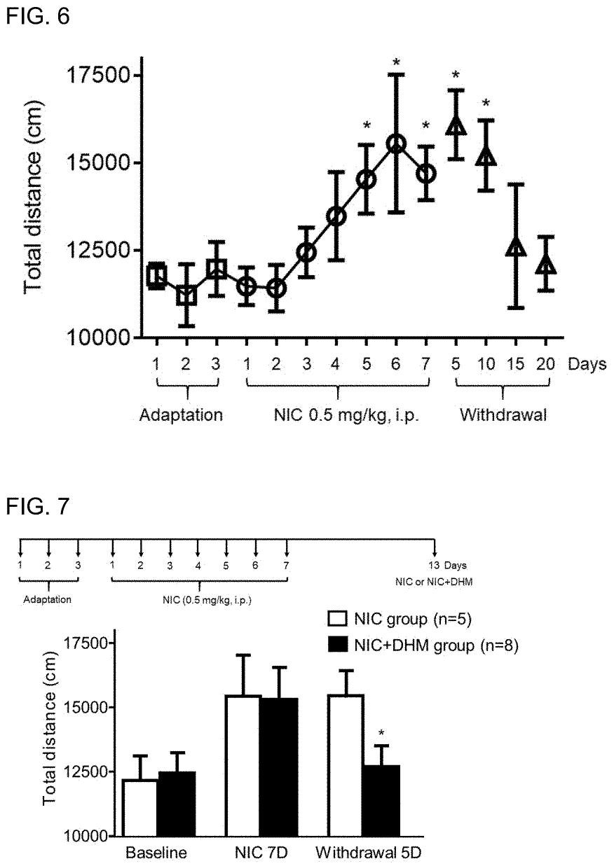

FIG. 7 shows effects of DHM on NIC-induced behavioral sensitization and a comparison of NIC sensitization between the NIC group and NIC+DHM group. The results demonstrated that DHM significantly reduced NIC-induced behavioral sensitization, and that DHM can be used to treat nicotine-related disorders and behaviors, such as smoking habits, and lead to smoking cessation.

FIGS. 1-7 show the effect of dihydromyricetin (DHM) on nicotinic acetylcholine receptor (nAChR) function using in vitro and in vivo electrophysiological recordings and animal behavioral measurements such as local motor activity (open field) and conditioned place preference (CPP). Such evidence provides substantiation that DHM can be used to treat nicotine-related disorders and behaviors, such as using DHM to reduce nicotine addition and lead to smoking cessation.

Specifically, as was shown in FIGS. 1-7. DHM inhibited a4b2-nAChR-mediated whole-cell currents in a concentration-dependent manner (FIG. 1). In mouse VTA slices, DHM reduced dopamine (DA) neuron firing rate and prevented nicotine-induced firing increase (FIG. 2). In anesthetized mice, DHM also abolished nicotine-induced DA neuronal firing increase (FIGS. 3-4). These electrophysiological experiments demonstrate that DHM inhibits a4b2-nAChR function. In animal behavioral experiments, DHM inhibits both the nicotine-induced CPP and local motor activity (FIGS. 5-7). Therefore, these experiments show that DHM inhibits nicotine receptor function and nicotine reward/dependence behaviors. Thus, DHM compositions will be useful in the treatment of nicotine addiction, including to help with smoking reduction and/or cessation.

### Example 4

This example provides a composition and method for using such for treating tobacco addiction or nicotine addiction, palliating nicotine withdrawal symptoms or facilitating smoking cessation comprising a therapeutically effective amount of DHM.

In alternative embodiments of this example, the DHM is used in a therapeutically effective combination with either an anti-depressant or an anti-anxiety drug. The anti-depressant is selected from the group consisting of bupropion, doxepin, desipramine, clomipramine, imipramine, nortriptyline, amitriptyline, protriptyline, trimipramine, fluoxetine, fluvoxamine, paroxetine, sertraline, phenelzine, tranylcypromine, amoxapine, maprotiline, trazodone, venlafaxine, mirtazapine, their pharmaceutically active salts and their optical isomers, anti-depressant is either bupropion or a pharmaceutically acceptable salt thereof, or doxepin or a pharmaceutically acceptable salt thereof.

In this example, the DHM or a pharmaceutically acceptable salt thereof, whether alone or formulated with another drug, is formulated to deliver a daily dose of DHM of about 1 mg to about 300 mg, and more preferably between about 1 mg and about 25 mg, and most preferably between about 1 mg and about 10 mg. A person skilled in the art can determine that other dosage amounts are to be used.

### Example 5

This example provides a method of providing DHM (e.g., selling and/or using) to (or with) a person, animal or an organization concerned with such (e.g., interest group, internet sales, pharmacy, doctor or veterinarian, patient). The method comprises formulating the DHM, or having the DHM formulated, in an orally available form selected from the group consisting of tablet, gummy, capsule (e.g., hard gelatin capsule, soft gelatin capsule, pullulan capsule, enteric coated capsule or hydroxypropyl methylcellulose capsule) or other orally available form known to a person of skill in the art; and facilitating access (e.g., making a statement on packaging, a virtual or brick and mortar store sign or statement, a brochure or other sales piece, a video, an article or a link to an article) to information concerning the use of DHM to treat a condition and/or cause an effect selected from the group consisting of tobacco addiction, nicotine addiction, palliating nicotine withdrawal symptoms, inhibiting nicotine reward, inhibiting nicotine dependence behaviors, inhibiting nicotine receptor function, reducing nicotine patch use, or facilitating cessation of smoking.

### Example 6

In this example, a patient was administered a pharmaceutical composition of this invention to treat cigarette smokers in order to reduce their cravings after they had stopped smoking. It was observed that in this patient, they reported improved quality and/or quantity of sleep.

### OTHER EMBODIMENTS

Although the present invention has been described with reference to teaching, examples and preferred embodiments, one skilled in the art can easily ascertain its essential characteristics, and without departing from the spirit and scope thereof can make various changes and modifications of the invention to adapt it to various usages and conditions. Those skilled in the art will recognize or be able to ascertain using no more than routine experimentation, many equivalents to the specific embodiments of the invention described herein. Such equivalents are encompassed by the scope of the present invention.

All publications, patents, and applications mentioned in this specification are herein incorporated by reference.

What is claimed is:

1. A method of treating nicotine addiction in a person or animal, comprising administering a therapeutically effective amount of dihydromyricetin, or a pharmaceutically acceptable salt thereof, to the person or animal.

2. The method of claim 1, wherein said dihydromyricetin is in a pharmaceutical composition that comprises a pharmaceutically acceptable carrier.

3. The method of claim 1, wherein said dihydromyricetin is incorporated into a sustained release preparation.

4. The method of claim 1, wherein the sleep of the person or animal is improved.

5. A method of reducing the desire to smoke, vape, nicotine patch, or chew tobacco in a person or animal, comprising administering a therapeutically effective amount of dihydromyricetin, or a pharmaceutically acceptable salt thereof, to the person or animal.

6. The method of claim 4, wherein said said dihydromyricetin is in a pharmaceutical composition that comprises a pharmaceutically acceptable carrier.

7. The method of claim 4, wherein said dihydromyricetin is incorporated into a sustained release preparation.

8. The method of claim 5, wherein the sleep of the person or animal is improved.

9. A method of inhibiting nicotine receptor function in a person or animal, comprising administering a therapeutically effective amount of dihydromyricetin, or a pharmaceutically acceptable salt thereof, to the person or animal.

10. The method of claim 7, wherein said dihydromyricetin is in a pharmaceutical composition that comprises a pharmaceutically acceptable carrier.

11. The method of claim 7, wherein said dihydromyricetin is incorporated into a sustained release preparation.

12. The method of claim 9, wherein the sleep of the person or animal is improved.

13. A method of inhibiting nicotine reward and/or dependence behaviors in a person or animal, comprising administering a therapeutically effective amount of dihydromyricetin, or a pharmaceutically acceptable salt thereof, to the person or animal.

14. The method of claim 10, wherein said dihydromyricetin is in a pharmaceutical composition that comprises a pharmaceutically acceptable carrier.

15. The method of claim 10, wherein said dihydromyricetin is incorporated into a sustained release preparation.

16. The method of claim 13, wherein the sleep of the person or animal is improved.

17. A method for treating tobacco addiction or nicotine addiction, palliating nicotine withdrawal symptoms or facilitating smoking cessation comprising administering a therapeutically effective amount of dihydromyricetin.

18. A method for treating, inhibiting and/or reducing the effects of nicotine and/or a symptom of nicotine withdrawal in a person or animal, comprising administering dihydromyricetin to the patient or animal.

19. A method for improving the quality of sleep of a person or animal, comprising administering dihydromyricetin to the patient or animal.

20. A method for improving the quantity of sleep of a person or animal, comprising administering dihydromyricetin to the patient or animal.

21. A method for providing dihydromyricetin to a person, animal or organization comprising,
(a) formulating the dihyromyricetin, or having the dihydromyricetin formulated, in an orally available form selected from the group consisting of tablet, gummy, hard gelatin capsule, soft gelatin capsule, pullulan capsule, enteric coated capsule or hydroxypropyl methylcellulose capsule; and
(b) facilitating access to information concerning the use of dihydromyricetin to treat a condition and/or cause an effect selected from the group consisting of tobacco addiction, nicotine addiction, palliating nicotine withdrawal symptoms, inhibiting nicotine reward, inhibiting nicotine dependence behaviors, inhibiting nicotine receptor function, reducing nicotine patch use, or facilitating cessation of smoking.

22. The method of claim 21, further comprising,
(c) facilitating access to information concerning the use of dihydromyricetin to improve the quality and/or quantity of sleep.
